(19) United States
(12) Patent Application Publication
(10) Pub. No.: US 2015/0119656 A1
(43) Pub. Date: Apr. 30, 2015

(54) Sensor Sheet And Monitoring System

(71) Applicant: Scott Foster, Ardmore, OK (US)

(72) Inventor: Scott Foster, Ardmore, OK (US)

(21) Appl. No.: 14/527,346

(22) Filed: Oct. 29, 2014

Related U.S. Application Data
(60) Provisional application No. 61/896,934, filed on Oct. 29, 2013.

(51) Int. Cl. A61B 5/00; A61B 5/145; A61B 5/107; A61F 13/42; A61B 5/01

(52) U.S. Cl. CPC A61B 5/0022; A61F 13/42; A61B 5/01; A61B 5/1075; A61B 5/145; A61B 5/6892
USPC 600/301; 604/361

(57) ABSTRACT

A medical sensing device contained in a sheet or mattress pad. The sensing device comprises a number of sets of sensors, each equipped for detecting a characteristic of a patient proximate the device. The characteristic is sent to a processor which determines whether the characteristic is within a pre-determined parameter. If not, an alarm is sounded.

### CROSS REFERENCE TO RELATED APPLICATION

This application claims the benefit of provisional patent application Ser. No. 61/896,934 filed on Oct. 29, 2013, the entire contents of which are incorporated herein by reference.

### FIELD

This invention relates generally to medical monitors and more specifically to a medical monitoring system for patients and infants. More particularly, this invention relates to integrating a passive sensor array into a mattress, a mattress pad, or sheet placed under a patient, child, or infant.

### SUMMARY

The present invention is directed to a medical sensing device. The device comprises a sheet, a set of first sensors spaced apart about the sheet, a set of second sensors spaced apart about the sheet, and a processor for receiving the first characteristic and second characteristic and generating an alert signal when the first characteristic or the second characteristic is outside a fixed parameter. The first sensors detect a first characteristic and the second sensors detect a second characteristic.

### BRIEF DESCRIPTION OF THE DRAWINGS

FIG. 1 an embodiment sensor sheet with a plurality of sensor arrays.

FIG. 2 is an embodiment sensor sheet with a plurality of sensor arrays with sensors coupled to a wiring harness.

### DETAILED DESCRIPTION

The present invention is described with reference to the attached figures, wherein like reference numerals are used throughout the figures to designate similar or equivalent elements. The figures are not drawn to scale and they are provided merely to illustrate the invention. Several aspects of the invention are described below with reference to example applications for illustration. It should be understood that numerous specific details, relationships, and methods are set forth to provide an understanding of the invention. One skilled in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

Sensors may detect a characteristic, for example, sensors that detect motion, pressure, temperature, pH, moisture, and other conditions are known to those that are skilled in the art.

One version of these sensors is to connect them to a computer or other such sensor reading device with wires. Another version is for the sensors to send data wirelessly to a sensor reading device via WiFi or Bluetooth.

The wireless sensors may be powered by an internal battery or may be powered externally by RF when interrogated by the sensor reading device in a manner similar to RFID tags.

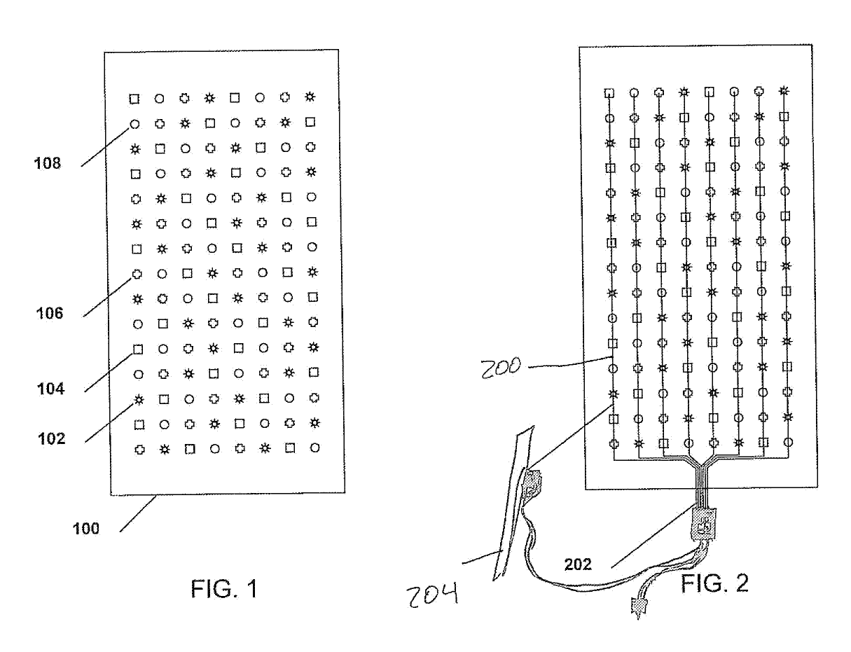

FIG. 1 illustrates and embodiment sheet 100 with an array of attached sensors 102, 104, 106, and 108. Each sensor 102, 104, 106, and 108 is adapted to detect a characteristic. A first sensor 102 may be a weight sensor to detect the presence and position of a child, infant or patient. A second sensor 104 may be a moisture sensor to detect blood or excretion of fluids. A third sensor 106 may be a pH sensor to differentiate between various body fluids. A fourth sensor 108 may be a temperature sensor. While these are listed, one of ordinary skill can appreciate that the positions of the sensors 102, 104, 106, 108 are not limiting and that other sensors, such as microphones, heartbeat, pulse, O₂ level, and lung monitors, etc. may be utilized.

Although a sheet 100 with arrays of sensors is illustrated in FIG. 1, the sheet may be the surface of a mattress, a mattress pad, or a pad under the bed sheet on which the patient, child, or infant is lying.

The sensors in FIG. 1 may communicate with a sensor reading device wirelessly with WiFi or Bluetooth. The sensors may be self-powered with an internal battery or may be externally powered by RF in a manner similar to RFID tags or powered by POE interne switch.

FIG. 2 illustrates an embodiment where the various sensors in the sensor arrays are connected by wires 200 to a wire harness 202 which may then be attached to a sensor reading device with a processor such as a computer 204. As shown the sensors are connected by a wired connection to the computer. One skilled in the art will appreciate that a wireless connection may be made to the computer, and that the computer may be located remotely from the sheet 100. One skilled in the art will appreciate that the sensors can be utilized to provide alarms for serious conditions, such as SIDS in infants or discrepancies, such as large variations in weight indicative of unauthorized persons in the bed or extended lack of movement. Data collected can be utilized to create reports, analyze historical data, sleep patterns and other medical data meaningful to one skilled in the art.

One embodiment for using the sheet is in a bed in a hospital. In this embodiment the sensor sheet 100 is connected to a sensor reading device such as a computer. The sensor reading device may be in the same room as the patient or infant or may be in another room or building. The sensor reading device may summarize the sensor data and plot it on graph. The sensor reading device may also send an alert to a nurse or parent if any of the sensor data goes out of a predetermined range. For example a cell phone alert could be sent out if the patient or infant or child gets out of the bed or fails to move for a certain length of time or wets the bed. One skilled in the art will appreciate that data gathered by the sheet 100 may include sleep positions, bodily fluid alerts, biometric statistics such as heart rate, oxygen level, and blood pressure, patterning data from patient history, breathing recognition and monitoring, absence alerts, weight notifications, contact point notification for non-authorized lifting of body parts, sleepwalker notification, changes in temperature, seizure recognition, shock notification for identification of abuse. Indicia such as the above may be used to provide automated alerts to caregivers or responding agencies, and historical data may be stored onsite or in the cloud. In addition, the doctor, nurse, or parent may receive an alert at any time regarding a change in status of the patient, child, or infant. A central interface for multiple sensor sheets 100 may be utilized at, for example, a nurses station in a hospital or medical practice group. Database information may also be utilized for more long-term analysis of data in nursing homes or hospice settings.

One skilled in the art will appreciate that the sensors in the sensor sheet 100 may communicate wirelessly with the sensor monitoring device. An interface for accessing the data may be provided on a remote computer, cell phone, or tablet computer. Further, the sheet 100 may comprise multiple components, for example, a sheet for detecting moisture and a pad for detecting pressure, temperature, etc.

While various embodiments of the present invention have been described above, it should be understood that they have been presented by way of example only and not limitation. Numerous changes to the disclosed embodiments can be made in accordance with the disclosure herein without departing from the spirit or scope of the invention. Thus, the breadth and scope of the present invention should not be limited by any of the above described embodiments.

What is claimed is:

1. A medical sensing device comprising:
a sheet;
a set of first sensors spaced apart about the sheet;
a set of second sensors spaced apart about the sheet;
wherein the first sensors detect a first characteristic and the second sensors detect a second characteristic;
a processor for receiving the first characteristic and second characteristic and generating an alert signal when the first characteristic or the second characteristic is outside a fixed parameter.

2. The medical sensing device of claim 1 wherein the set of first sensors detect moisture.

3. The medical sensing device of claim 2 wherein the set of second sensors detect temperature.

4. The medical sensing device of claim 2 wherein the set of first sensors distinguishes between moisture comprising blood and moisture comprising urine.

5. The medical sensing device of claim 1 wherein the sheet comprises a blanket.

6. The medical sensing device of claim 1 wherein the sheet comprises a mattress pad.

7. The medical sensing device of claim 1 further comprising a wire harness connected to the sheet and the processor.

8. The medical sensing device of claim 1 wherein the first characteristic and second characteristic are received wirelessly at the processor.

9. The medical sensing device of claim 8 wherein the processor is located at a nurse station.

10. The medical sensing device of claim 8 wherein the alert signal is generated at a cell phone.

11. The medical sensing device of claim 1 further comprising a set of third sensors to detect a third characteristic.

12. The medical sensing device of claim 11 further comprising a set of fourth sensors to detect a fourth characteristic.

13. The medical sensing device of claim 11 wherein the third characteristic comprises pressure.

14. A method for detecting a condition in a patient comprising:
providing a sheet having a plurality of sensors;
detecting at least two characteristics at the sheet;
transmitting a signal based upon the at least two characteristics to a processor;
determining whether the at least two characteristics are within a pre-set parameter; and
triggering an alarm is the at least two characteristics are not within a pre-set parameter.
